(19) United States
(12) Patent Application Publication
(10) Pub. No.: US 2021/0338952 A1
(43) Pub. Date: Nov. 4, 2021

(54) TWO PNEUMATIC CYLINDER MEDICAL VENTILATOR, SYSTEM AND METHOD

(71) Applicant: Groman Inc., Margate, FL (US)

(72) Inventor: Boaz Barry Groman, Boca Raton, FL (US)

(21) Appl. No.: 17/246,741

(22) Filed: May 3, 2021

Related U.S. Application Data
(60) Provisional application No. 63/018,518, filed on May 1, 2020.

(51) Int. Cl. A61M 16/00; A61M 16/20

(52) U.S. Cl. CPC A61M 16/0072; A61M 16/201; A61M 16/208

(57) ABSTRACT

A medical ventilator comprising two pistons moving in unison with one another within respective two (a “first” and a “second”) cylinders. During an exhale phase of a breathing cycle, the first cylinder receives pressurized air which causes both pistons to move (upward). In the second cylinder, this creates a negative pressure to extract exhaled air from a patient's lungs. During an inhale phase of the breathing cycle, a weight acting on a link between the two pistons causes both pistons to move in an opposite (downward) direction, whereupon (i) the first piston delivers the pressurized air to the patient and (ii) the second piston vents the exhaled air. The second cylinder may have a larger bore or a larger stroke than the first cylinder, or may comprise multiple (a bank of) smaller cylinders.

### CROSS REFERENCE TO RELATED APPLICATIONS

This is a nonprovisional filing of U.S. 63/018,518 filed 1 May 2020.

### FIELD OF THE INVENTION

This invention relates to (medical) ventilators. Generally, a ventilator is a machine that provides mechanical ventilation by moving breathable air into and out of the lungs, to deliver breaths to a patient who is physically unable to breathe, or breathing insufficiently.

### BACKGROUND

The process of breathing (respiration) is divided into two distinct phases, inspiration (inhalation) and expiration (exhalation). It is a mechanical process that depends on volume changes in the chest cavity. The volume changes result in pressure changes, which lead to the flow of gases to equalize the pressure. At the start of a breath, pressure inside and outside the lungs is equal. Normally about 500 ml (1 pint) of air is moved in and out per breath—this is known as the tidal volume.

A ventilator is a machine that provides mechanical ventilation by moving breathable air into and out of the lungs, to deliver breaths to a patient who is physically unable to breathe, or breathing insufficiently. Modern ventilators are computerized microprocessor controlled machines, but patients can also be ventilated with a simple, hand-operated bag valve mask. Ventilators are chiefly used in intensive care medicine, home care, and emergency medicine (as standalone units) and in anesthesiology (as a component of an anesthesia machine).

Ventilators are sometimes called respirators, a term commonly used for them in the 1950s (particularly the “Bird respirator”). However, in contemporary hospital and medical terminology, a respirator is a protective face mask.

A ventilator's basic function is to drive a specified volume of gas—air, pure oxygen, or combination of both—into the patient's lungs and withdraw the same volume of gas from the lungs to remove CO2. The volume of gas driven into the lungs is called Tidal Volume and is specific for each patient based on size and health of lungs. Normal Tidal Volume range is 200-800 ml and is usually calculated based on patient's weight at 10-15 ml/Kg. The volume of gas is injected into the lungs at low pressure usually 0.50-1 psi. Slightly higher initial pressure may be required for patients with damaged lungs, but pressure can not be high as it may damage the lungs.

Breathable gas—air, pure oxygen, or combination of both—is available via portable pressurized gas tanks via through port connection available at hospitals. Hospital oxygen supply normally provides 50-55 psi.

In its simplest form, a modern positive pressure ventilator consists of a compressible air reservoir or turbine, air and oxygen supplies, a set of valves and tubes, and a disposable or reusable “patient circuit”. The air reservoir is pneumatically compressed several times a minute to deliver room-air, or in most cases, an air/oxygen mixture to the patient. If a turbine is used, the turbine pushes air through the ventilator, with a flow valve adjusting pressure to meet patient-specific parameters. When over pressure is released, the patient will exhale passively due to the lungs' elasticity, the exhaled air being released usually through a one-way valve within the patient circuit called the patient manifold.

Ventilators may also be equipped with monitoring and alarm systems for patient-related parameters (e.g. pressure, volume, and flow) and ventilator function (e.g. air leakage, power failure, mechanical failure), backup batteries, oxygen tanks, and remote control. The pneumatic system is nowadays often replaced by a computer-controlled turbopump.

Modern ventilators are electronically controlled by a small embedded system to allow exact adaptation of pressure and flow characteristics to an individual patient's needs. Fine-tuned ventilator settings also serve to make ventilation more tolerable and comfortable for the patient. In Canada and the United States, respiratory therapists are responsible for tuning these settings, while biomedical technologists are responsible for the maintenance. In the United Kingdom and Europe the management of the patient's interaction with the ventilator is done by critical care nurses.

The patient circuit usually consists of a set of three durable, yet lightweight plastic tubes, separated by function (e.g. inhaled air, patient pressure, exhaled air). Determined by the type of ventilation needed, the patient-end of the circuit may be either noninvasive or invasive.

Noninvasive methods, such as continuous positive airway pressure (CPAP) and non-invasive ventilation, which are adequate for patients who require a ventilator only while sleeping and resting, mainly employ a nasal mask. Invasive methods require intubation, which for long-term ventilator dependence will normally be a tracheotomy cannula, as this is much more comfortable and practical for long-term care than is larynx or nasal intubation.

### Open Source Ventilators

A number of “open source” ventilators are being developed, and there are some concerns/issues. Refer to https://f1000research.com/articles/9-218

One challenge is maintaining a proper level of sterility of devices fabricated using distributed means. Specifically, for fused filament fabrication (FFF) based 3-D printing parts, it has been reported that the prints are sterile at the time of print. If not kept in a sterile environment, however, they could quickly become biologically contaminated. One approach to deal with this is to use washing or a chemical bath. If a specific polymer is needed that cannot be 3-D printed easily, it is possible to make molds in high-temperature plastics, such as polycarbonate, and then use lower temperature plastics to make disposable single use plastic parts. Similarly, silicone molds can be made from a 3-D printed reverse mold and used in the same way.

For medical professionals to use an open source ventilator, they first must be convinced it will do no harm to them (or others) as well as to the patient. As COVID-19 was reported to spread via droplets, contact and natural aerosols from human-to-human, there has been a concern that high-risk aerosol-producing procedures may put medical personnel at high risk of nosocomial (originating in a hospital) infections.

There is clear technical potential for alleviating ventilator shortages during the current COVID19 pandemic, as well as for future pandemics, and for everyday use in low-resource settings using open source ventilator designs that can be rapidly fabricated using distributed manufacturing.

### Bird Ventilator

Forrest M. Bird is the inventor of the first convenient and reliable, low cost, mass-produced medical respirator, referred to as a medical ventilator in Bird's U.S. Pat. No. 3,842,828. Bird also helped reduce infant mortality rates in babies with respiratory issues with his invention dubbed the “Babybird Respirator” whose technology traces back to U.S. Pat. No. 3,191,596.

Bird was born in Stoughton, Mass. Bird became a pilot at an early age due to the encouragement of his father, a World War I pilot, and from meeting Orville Wright at an early age. He performed his first solo flight at age 14. By age 16 he was working to obtain multiple major pilot certifications.[2] Bird enlisted with the United States Army Air Corps, and entered active duty in 1941 as a technical air training officer due to his advanced qualifications. This rank, combined with the onset of World War II, gave him the opportunity to pilot almost every aircraft in service, including early jet aircraft and helicopters.

https://en.wikipedia.org/wiki/Forrest_Bird

U.S. Pat. No. 3,191,596 (1965 Jun. 29; Bird et al.) discloses a respirator. As claimed therein,
1. In a respiratory apparatus having an inhalation phase and an exhalation phase in its operative cycle, a controller having an inlet adapted to be connected to a source of gas under pressure, said controller having an outlet adapted to be connected to the airway of the patient, a main control valve in the controller movable between open and closed positions to control the flow of gas from the inlet to the outlet, means for operating said main control valve so that the main control valve is in an open position during the inhalation phase and in a closed position during the exhalation phase of the respiratory apparatus, said means including means for sensing when a predetermined pressure is reached in the outlet for shifting said main control valve from an open position to a closed position, a fluid source, valve means connected to the outlet for sensing when the predetermined pressure is not reached in the outlet in a predetermined time, and additional valve means controlled by said first named valve means for introducing additional gas into the outlet to cause the pressure in said outlet to reach the predetermined pressure, said first named valve means controlling the application of fluid from said fluid source to said additional valve means to cause operation of said additional valve means.

U.S. Pat. No. 3,842,828 (1974 Oct. 22; Bird) discloses pediatric ventilator having an inhalation phase and an exhalation phase in its operative cycle with an inlet adapted to be connected to a source of gas under pressure. A breathing circuit is adapted to be connected to the patient. Nebulizing means is provided. Flow divider means is connected to the inlet and has one outlet coupled to the nebulizing means so that at least a portion of the inlet gas is supplied to the nebulizing means. The flow divider means includes an additional outlet coupled to the breathing circuit and has means for controlling the flow of gas through the additional outlet whereby precise control over nebulization can be obtained.

U.S. Pat. No. 3,915,164 (1975 Oct. 28; Bird) discloses ventilator with an inhalation phase and an exhalation phase in its operative cycle having a servo controller with an inlet adapted to be connected to supply gas under pressure and also having an outlet. The controller has control valve means movable between open and closed positions to control the flow of gas from the inlet to the outlet of the servo controller. The control valve means is an open position during the inhalation phase of the ventilator and in a closed position in the exhalation phase of the ventilator. Means is provided for supplying gases to the patient from the servo controller until a predetermined pressure has been reached. After the predetermined pressure is reached, means is provided to supply an additional flow of gases to the patient to provide an inspiratory apneustic plateau for the patient. After a predetermined period of time, the patient is permitted to exhale and thereafter the same cycle is repeated.

### Some More Patents by Bird

U.S. Pat. No. 8,347,883 (2009 Apr. 10; Bird) discloses manual controlled bi-phasic intrapulmonary percussive ventilation and methods. The method and system ventilates a patient's airway during the inspiratory phase and expiratory phase from a source of pressurized gas, typically from a compressor. The system and method supplies, to the patient airway during the inspiratory phase, a plurality of pulses of small volumes of gas from the gas source, and adds, in succession, pulses of small volumes of gas to provide successively greater volumes of gas successively increasing in pulsatile form the pressure of the gas in the patient's airway. This addition of successively greater volumes of gas serves to provide diffusive ventilation to the patient during the inspiratory phase, and, permits the patient to exhale during the expiratory phase.

US 2010/0125227 (2010 May 20; Bird) discloses Manual Controlled Bi-Phasic Intrapulmonary Percussive Ventilation and Methods. The method and system ventilates a patient's airway during the inspiratory phase and expiratory phase from a source of pressurized gas, typically from a compressor. The system and method supplies, to the patient airway during the inspiratory phase, a plurality of pulses of small volumes of gas from the gas source, and adds, in succession, pulses of small volumes of gas to provide successively greater volumes of gas successively increasing in pulsatile form the pressure of the gas in the patient's airway. This addition of successively greater volumes of gas serves to provide diffusive ventilation to the patient during the inspiratory phase, and, permits the patient to exhale during the expiratory phase.

U.S. Pat. No. 6,595,203 (2003 Jul. 22; Bird) discloses Apparatus for administering intermittent percussive ventilation and unitary breathing head assembly for use therein. A unitary breathing head device for administering intermittent percussive ventilation to a patient having an airway and for use with an IPV device having a source of continuous gas flow and having a source of pulsed gases comprising a combination injector and exhalation valve assembly comprising a main body having an airway port for communication with the airway of the patient and having proximal and distal extremities and a flow passage extending from the proximal extremity to the distal extremity. The main body has a depending portion forming a plenum chamber in communication with the flow passage in the main body. A nebulizer is removably secured to the depending portion of the main body and has a nebulizer chamber in communication with the plenum chamber. The depending portion of the main body and the nebulizer form a handle adapted to be gripped by the hand of the patient holding the breathing head assembly.

U.S. Pat. No. 6,581,600 (2003 Jun. 24; Bird) discloses Interface apparatus and combination and method. Interface apparatus for use by a patient having a patient airway and for use with a mechanical ventilator having inspiratory and expiratory tubing. An intrapulmonary percussive ventilator having an output and an injection device coupled to the output of the intrapulmonary percussive ventilator and having an output. The interface apparatus comprises a mixing device having a mixing chamber therein and having an outlet adapted to be connected to the patient airway. The body has fittings in communication with the mixing chamber adapted to be connected to the inspiratory tubing and to the expiratory tubing of the mechanical ventilator. The body also has an injection port in communication with the mixing chamber adapted to be connected to the output of the injection device.

US 2003/0010344 (2003 Jan. 16; Bird) discloses interface apparatus for use with a continuous mechanical volume ventilator and an intrapulmonary percussive ventilator and combination thereof and method. Interface apparatus for use by a patient having a patient airway and for use with a mechanical ventilator having inspiratory and expiratory tubing. An intrapulmonary percussive ventilator having an output and an injection device coupled to the output of the intrapulmonary percussive ventilator and having an output. The interface apparatus comprises a mixing device having a mixing chamber therein and having an outlet adapted to be connected to the patient airway. The body has fittings in communication with the mixing chamber adapted to be connected to the inspiratory tubing and to the expiratory tubing of the mechanical ventilator. The body also has an injection port in communication with the mixing chamber adapted to be connected to the output of the injection device.

U.S. Pat. No. 5,862,802 (1999 Jan. 26; Bird) discloses ventilator having an oscillatory inspiratory phase and method. Ventilator for use with a source of gas under pressure for supplying such gas to the airway of a patient having an inlet adapted to be connected to the source of gas, and an outlet adapted to be connected to the airway of the patient. A pneumatic oscillator is connected to the inlet for supplying pulsatile gas in the form of successive small volumes of gas to the airway of the patient during a breath of the patient to cause diffusive ventilation of the airway to the patient. An exhalation valve assembly is connected to the patient airway for permitting the patient to exhale gases introduced into the patient airway.

U.S. Pat. No. 5,165,398 (1992 Nov. 24; Bird) discloses ventilator and oscillator for use therewith and method. A ventilator in combination with a pneumatic oscillator cartridge comprising a body with an inlet, an outlet, a diaphragm operated valve, and a servo port. An adjustable valve is provided for metering the flow of gas from the outlet to the servo port to provide cyclic operation of the oscillator cartridge. A pneumatic clutching assembly is provided which has an inlet and an outlet. The outlet of the oscillator cartridge is connected to the inlet of the pneumatic clutching assembly. An oscillator canister is provided which has an inlet and an outlet. The outlet of the pneumatic clutching means is coupled to the inlet of the oscillator canister. The oscillator canister has a shaft, a diaphragm mounted on the shaft and separating the canister into first and second chambers. Countersprings are mounted on the shaft. The oscillator canister serves to supply and withdraw small volumes of gas to and from the patient adaptor.

U.S. Pat. No. 5,116,088 (1992 May 26; Bird) discloses ventilator having an oscillatory inspiratory phase and method. Quick disconnect assembly comprising a female fitting having a socket formed therein and a male fitting having a bayonet adapted to fit in said socket. Seals are carried by the female and male fittings for forming an airtight seal between the bayonet and the socket. A retainer is provided for retaining said fittings in engagement with each other.

U.S. Pat. No. 5,007,420 (1991 Apr. 16; Bird) discloses ventilator having an oscillatory inspiratory phase and method. Ventilator for use with a source of gas under pressure for supplying such gas to the airway of a patient having an inlet adapted to be connected to the source of gas, and an outlet adapted to be connected to the airway of the patient. A pneumatic oscillator is connected to the inlet for supplying pulsatile gas in the form of successive small volumes of gas to the airway of the patient during a breath of the patient to cause diffusive ventilation of the airway to the patient. An exhalation valve assembly is connected to the patient airway for permitting the patient to exhale gases introduced into the patient airway.

U.S. Pat. No. 4,930,501 (1990 Jun. 5; Bird) discloses ventilator adapted to be connected to a source of gas comprising a case and an inlet adapted to be connected to the source of gas. It also is comprised of an oscillator cartridge carried by the case having a body with an inlet and an outlet and a flow passage interconnecting the inlet and the outlet. A valve member is carried by the body and is movable between open and closed positions with respect to the outlet. A diaphragm is also carried by the body and is connected to the valve member. A servo port is carried by the body for supplying gas to one side of the diaphragm to cause movement of the diaphragm to move the valve member between open and closed positions to interrupt the flow of gas from the inlet to the outlet. An adjustable impact metering valve is provided for metering the flow of gas to the servo port. A patient adapter and a pneumatic clutching device having an output coupled to the airway of the patient adapter are provided.

U.S. Pat. No. 4,867,151 (1989 Sep. 19; Bird) discloses mobile self-contained ventilator having a framework and a compressor carried by said framework. The compressor has a motor and at least one fan driven by the motor which causes air to pass over the motor for cooling the motor and for picking up heat from the motor. The compressor also has an output through which compressed air is supplied. A respirator is carried by the framework and includes at least one oscillator cartridge having an inlet connected to the outlet for receiving compressed air from the outlet. The oscillator cartridge is disposed so that air, after it has passed over the motor, passes over the cartridge to heat the cartridge to inhibit moisture condensation within the cartridge.

U.S. Pat. No. 4,838,260 (1989 Jun. 13; Bird) discloses ventilator adapted to be connected to a source of gas comprising a case and an inlet adapted to be connected to the source of gas. It also is comprised of an oscillator cartridge carried by the case having a body with an inlet and an outlet and a flow passage interconnecting the inlet and the outlet. A valve member is carried by the body and is movable between open and closed positions with respect to the outlet. A diaphragm is also carried by the body and is connected to the valve member. A servo port is carried by the body for supplying gas to one side of the diaphragm to cause movement of the diaphragm to move the valve member between open and closed positions to interrupt the flow of gas from the inlet to the outlet. An adjustable impact metering valve is provided for metering the flow of gas to the servo port. A patient adapter and a pneumatic clutching device having an output coupled to the airway of the patient adapter are provided.

U.S. Pat. No. 4,805,613 (1989 Feb. 21; Bird) discloses ventilator which can be readily transported for emergency situations. Ventilator adapted to be connected to a source of gas under pressure comprising a case and an inlet adapted to be connected to the source of gas. It also is comprised of an oscillator cartridge carried by the case and having a body with an inlet and an outlet and a flow passage interconnecting the inlet and the outlet. A valve member is carried by the body and is movable between open and closed positions with respect to the outlet. A diaphragm is carried by the body and is connected to the valve member for moving the valve member between and open and closed positions to interrupt the flow of gas in the flow passage between the inlet and the outlet. A servo port is carried by the body for supplying gas to the diaphragm for causing movement of the diaphragm to thereby cause movement of the valve member to the closed position to interrupt the flow of gas in the flow passage between the inlet and the outlet.

U.S. Pat. No. 4,742,823 (1988 May 10; Bird) discloses liquid injector for wetting mechanically delivered intrapulmonary gasses having a reservoir adapted to contain a quantity of liquid. A tube extends downwardly into the reservoir to a level below the level of the liquid in the reservoir. A one-way check valve controls the flow of liquid from the tube. Tubing including an additional one-way check valve is provided for delivering gas under pressure to the reservoir above the liquid in the reservoir to apply pressure to the liquid in the reservoir to force the liquid up through the tube. An orifice is provided which is in communication with the first named one-way check valve means. A body is provided forming a chamber surrounding the orifice. A valve is provided for adjusting the flow of liquid from the orifice. Gas under pressure is supplied to the chamber to cause the gas to come in contact with the liquid passing through the orifice means. Gas is withdrawn from the chamber after liquid has been introduced into the gas.

U.S. Pat. No. 4,592,349 (1986 Jun. 3; Bird) discloses ventilator having an oscillatory inspiratory phase and method. Ventilator for use with a source of gas under pressure for supplying such gas to the airway of a patient having an inlet adapted to be connected to the source of gas, and an outlet adapted to be connected to the airway of the patient. A pneumatic oscillator is connected to the inlet for supplying pulsatile gas in the form successive small volumes of gas to the airway of the patient during a breath of the patient to cause diffusive ventilation of the airway of the patient. An exhalation valve assembly is connected to the patient airway for permitting the patient to exhale gases introduced into the patient airway.

U.S. Pat. No. 4,197,843 (1980 Apr. 15; Bird) discloses a volume limiting ventilator having a sequencing servo for switching from an inhalation phase to an exhalation phase in its operative cycle. A bellows mounted in a container is provided for receiving gas from a source of gas under pressure. A master venturi is in communication with the interior of the bellows and the exterior of the bellows. A large air breathing tube forming a part of a breathing circuit connects the interior of the bellows to a patient adapter. A master flow cartridge controlled by the sequencing servo supplies source gas to the master venturi. A transfer valve assembly is provided for determining whether the gas flows straight through to the breathing circuit or alternatively is supplied to the container carrying the bellows to raise the bellows. The transfer valve assembly controls the transfer of gas in the container from the exterior of the bellows to the interior of the bellows.

U.S. Pat. No. 4,164,219 (1979 Aug. 14; Bird) discloses a ventilator which may be operated in either of two manually selected modes, including (1) a first mode in which ventilation of a patient is initiated by inhalation of the patient through a patient adapter and is terminated to allow the patient to exhale when the pressure at the patient adapter reaches a predetermined level; or (2) a second mode in which ventilation of the patient is initiated in the same manner as in the first mode, but in which apneustic hold means are activated when the pressure at the patient adapter reaches the predetermined level and maintains gas pressure and flow in the patient adapter for a short predetermined time thereafter before the patient is allowed to exhale.

U.S. Pat. No. 4,148,312 (1979 Apr. 10; Bird) discloses combination anesthesia and intensive care apparatus (unit) having an anesthesia apparatus in the form of an analgesia generator and a anesthesia respirator and with a vaporizer and CO.sub.2 absorber mounted thereon. A removable chair assembly is connected to the stand and is to be used by the anesthesiologist in operating the anesthesia apparatus for intensive care. A respirator and a patient breathing monitoring apparatus are also mounted upon the stand. When used as an intensive care unit, the chair assembly can be disconnected from the stand.

U.S. Pat. No. 4,148,313 (1979 Apr. 10; Bird et al.) Patient breathing monitoring apparatus (and method) used for monitoring the airway of a patient which has a respirator connected thereto and which is connected to a source of gas. The respirator is of a type which during the inhalation phase supplies inspiratory gases to the airway of the patient and during exhalation phase permits the discharge of gases from the airway of the patient. The monitoring apparatus includes first and second pressure switches each of which has an inlet connected to the airway of the patient. One of the pressure switches is adjustable to sense a decrease in pressure in the airway below a predetermined value whereas the other of the switches is adjustable to sense an increase in pressure in the airway above a predetermined value. An alarm devices is coupled to the pressure switches for giving an alarm when the one switch senses a decrease in pressure below the predetermined value and when the other of the switches senses an increase in pressure above a predetermined value.

U.S. Pat. No. 4,127,123 (1978 Nov. 28; Bird) discloses ventilator (and method) having an inhalation phase and an exhalation phase in its operative cycle for use with a source of gas under pressure. A master sequencing cartridge having an inlet adapted to be connected to a source of gas under pressure and an outlet is provided. The cartridge has a valve member movable between open and closed positions to control the flow of gas from the inlet to the outlet. The cartridge is provided with a diaphragm capable of operating under differentials in pressure for causing movement of said valve member. A breathing circuit outlet is provided and is coupled to the outlet of the master sequencing cartridge. A pneumatic control circuit is provided for controlling the movement of the valve member of the master sequencing cartridge between open and closed positions and includes a volume/rate control valve assembly having an inlet and an outlet.

U.S. Pat. No. 4,121,579 (1976 Aug. 9; Bird) discloses ventilator and method. A ventilator with an inhalation phase and an exhalation phase in its operative cycle having an inlet adapted to be connected to a supply of gas under pressure and first, second and third outlets. A servo controller is provided having an inlet and an outlet with control valve means movable between open and closed positions to control the flow of gas from the inlet to the outlet. Means is provided for supplying gas from the first outlet to the patient adapter. An exhalation valve assembly is coupled to the patient adapter and is movable between open and closed positions. Means is provided for supplying gas from the second outlet to the exhalation valve assembly to maintain the exhalation valve assembly in a closed position during the time the gas is being supplied from the outlet of the servo controller.

U.S. Pat. No. 4,080,103 (1978 Mar. 21; Bird) discloses portable air compressor system for respirator. Portable air compressor system for a respirator and adapted to be operated from a source of power. A plurality of compressors are provided. Each compressor has a fan for causing air to move over the compressor and has an outlet supplying compressed air. A cooling coil is provided having an inlet and an outlet and is positioned so that the fans of the compressors force air over the cooling coil. Piping connects the outlets of the compressors to the inlet of the cooling coil. A water trap is provided. Piping connects the outlet of the cooling coil to the water trap. An air reservoir is provided which has an inlet that connects it to the water trap and an outlet which is adapted to be connected to the respirator. A pressure regulator is provided which is in communication with the reservoir. A pressure switch is provided which is connected to the regulator and at least one of the compressors for halting operation of said at least one compressor when a predetermined pressure is reached in the reservoir.

U.S. Pat. No. 4,060,078 (1977 Nov. 29; Bird) discloses ventilator (and method) having an inhalation phase and an exhalation phase in its operative cycle for use with a source of gas under pressure. A demand flow accelerator is responsive to the pressure of the gases in a breathing head assembly and provides additional gases to the breathing head assembly when the pressure of the gases in the breathing head assembly falls below a predetermined pressure. A sensor is also provided responsive to the pressure of the gases in the breathing head assembly for supplying gases to the breathing head assembly when the pressure of the gases in the breathing head assembly falls below a predetermined value to cause the patient to exhale against a substantially constant positive airway pressure. An additional sensor is also provided which is sensitive to the airway pressure being sensed for bleeding gases from the breathing circuit when pressure greater than a predetermined pressure are reached.

U.S. Pat. No. 4,044,763 (1977 Aug. 30; Bird) discloses ventilator (and method) having an inhalation phase and an exhalation phase in its operative cycle for use with a source of gas under pressure. A master sequencing cartridge having an inlet adapted to be connected to a source of gas under pressure and an outlet is provided. The cartridge has a valve member movable between open and closed positions to control the flow of gas from the inlet to the outlet. The cartridge is provided with a diaphragm capable of operating under differentials in pressure for causing movement of said valve member. A breathing circuit outlet is provided and tubing is provided for coupling the breathing circuit outlet to the outlet of the master sequencing cartridge. A volume-rate control valve assembly is provided for controlling the movement of the valve member of the master sequencing cartridge between open and closed positions.

U.S. Pat. No. 4,039,139 (1977 Aug. 2; Bird) discloses ventilator (and method) with an inhalation phase and an exhalation phase in its operative cycle having a servo controller with an inlet adapted to be connected to supply gas under pressure and also having an outlet. The controller has control valve means movable between open and closed positions to control the flow of gas from the inlet to the outlet of the servo controller. The control valve means is an open position during the inhalation phase of the ventilator and in a closed position in the exhalation phase of the ventilator. Means is provided for supplying gases to the patient from the servo controller until a predetermined pressure has been reached. After the predetermined pressure is reached, means is provided to supply an additional flow of gases to the patient to provide an inspiratory apneustic plateau for the patient. After a predetermined period of time, the patient is permitted to exhale and thereafter the same cycle is repeated.

U.S. Pat. No. 4,037,994 (1977 Jul. 26; Bird) discloses pressure unloading valve device for compressor. A valve device is disclosed for unloading pressure from a compressor of the type utilized in a medical respirator. The device is formed with a cylindrical chamber containing a compressible elastomeric member. Inlet and relief ports of the valve open into the chamber, and the inlet port is connected with the outlet of the gas compressor. Bistable actuator means is provided for moving a plunger within the chamber which in turn causes the cylindrical member to assume either a compressed or uncompressed state. In its uncompressed state a radial clearance between the outer wall of the elastomeric member and the chamber provides a flow path between the inlet and relief ports to bleed pressure from the compressor. In its compressed state the elastomeric member expands to occlude the flowpath and prevent pressure bleed-off from the compressor.

U.S. Pat. No. 4,020,834 (1977 May 3; Bird) discloses respirator (and method) with an inhalation phase and an exhalation phase in its operative cycle having an inlet adapted to be connected to a supply of gas under pressure and first and second outlets. A device is provided for establishing a positive pressure above atmospheric against which the patient must exhale during the exhalation phase for a predetermined period near the end of the exhalation phase. A device is provided for terminating the application of positive pressure so that the patient is exposed to ambient atmospheric pressure prior to initiation of the inhalation phase. Inspiratory flow acceleration means is provided for supplying additional gases to the first outlet during the inhalation phase. A control device is provided for establishing the length of the inhalation phase and includes an auxiliary reservoir for collecting gas and means for bleeding off the gas from the auxiliary reservoir whereby the exhalation time can be adjusted without being adversely affected by the pressure of the inlet gas.

U.S. Pat. No. 3,984,133 (1976 Oct. 5; Bird) discloses a connector assembly for rapidly forming a secure fluid-tight connection in a fluid circuit. The proximal ends of the fittings are adapted for connection with tubing, hose or other fittings of the circuit. An annular connector cap is formed at its opposite ends with inwardly projecting detent shoulders. One detent shoulder is sized for engagement in an annular locking groove formed about the outer surface of the female fitting while the other detent shoulder is sized for locking engagement with one of a series of annular locking barbs formed about the male fitting. Inclined camming surfaces are associated with the barbs for camming the corresponding detent shoulder radially outwardly and thereby facilitate locking of the detent with a selected barb which securely holds the fittings together in fluid-tight relationship.

U.S. Pat. No. 3,974,828 (1976 Aug. 17; Bird) discloses a ventilator (and method) with an inhalation phase and an exhalation phase in its operative cycle having an inlet adapted to be connected to a supply of gas under pressure and first, second and third outlets. A servo controller is provided having an inlet and an outlet with control valve means movable between open and closed positions to control the flow of gas from the inlet to the outlet. A conduit system is provided for supplying gas from the first outlet to the patient adapter. An exhalation valve assembly is coupled to the patient adapter and is movable between open and closed positions. A conduit system is provided for supplying gas from the second outlet to the exhalation valve assembly to maintain the exhalation valve assembly in a closed position during the time the gas is being supplied from the outlet of the servo controller.

### Manley Ventilator

Roger Manley suggested the possibility of using the pressure of the gases from the anaesthetic machine as the motive power for a simple apparatus to ventilate the lungs of patients in the operating theatre. We discussed the principle of the Bird ventilator and the Stephenson pressure valve system used by the American Paramedical Services to administer oxygen for resuscitation. We puzzled as to whether the reduced gas pressure emerging from the Rotameters of an anaesthetic machine would suffice to drive a ventilator. By the following Monday, Roger had made a working prototype and demonstrated that the gas pressure was sufficient to fill a reservoir bag, raising a weight to a point where it opened a valve an triggered off the compression of the gas bag delivering its contents to the patient. It was a ‘mark 2’ version of this apparatus that he took to Blease who refined it and developed it as the Manley ventilator. With the advent of this ventilator a cheap, simple effective means of IPPV became available in every operating theatre. It soon replaced the ‘educated hand’ as a means of ventilating patients and hastened the acceptance of total paralysis and artificial ventilation rather than partial paralysis and assisted ventilation. See Anaesthesia, 1995, Volume 50, pages 6471

### Some Additional Background

The following are referenced, and may be incorporated by reference herein.

https://www.news4jax.com/health/2020/03/30/uf-researchers-develop-low-cost-open-source-ventilator/https://www.aast.org/GeneralInformation/mechanicalventilation.aspx

https://www.medgadget.com/2020/03/university-of-minnesota-develops-simpler-inexpensive-mechanical-ventilator.html

https://www.discovermagazine.com/technology/how-to-build-a-mechanical-ventilator-for-a-few-hundred-euros Cristiano Galbiati, at Princeton University, and a team of colleagues that spans the Americas and Europe, who have published the design of a mechanical ventilator that they have prototyped and built in just a few days and at minimal cost. The machine is designed to be mass-produced specifically to tackle the COVID-19 pandemic.

The team base their design on an old but reliable machine called a Manley Ventilator, originally developed in the 1950s by anesthetist Roger Manley. His key insight was to use the pressure of gas from an anesthetic machine to help his patients breathe. So his machine was entirely gas-driven. Later, engineers introduced various sensors and actuators to control and monitor what is going on. But the design is still extremely simple, requiring just a power supply and a gas supply.

Galbiati and colleagues say the main difference between theirs and Manley's machine is that they use electronic pneumatic valves that can be controlled by computer, rather than mechanical switches. This eases the move to large-scale production, they say.

The ventilator consists of a supply of oxygen or medical air under pressure; a flow meter to measure the flow of gas; a couple of pneumatic valves to control this flow and a pressure sensor and microcontroller to control the valves.

In addition, the machine maintains a specific gas pressure into and out of the lungs using the weight of a column of water in a simple device known as a vent trap. Lastly a device known as a spirometer measures the volume of gas expelled by the lungs on each breath, to monitor breathing patterns.

The ventilator can also operate in an assisted mode where it senses the pressure change associated with the first stage of a patient's breath and then kicks in to help.

And that's it. The machine is called the Mechanical Ventilator Milano and all its components are readily available off the shelf. “The total cost of components is a few hundreds of euros,” say the team. They have even ensured it meets the U.K.'s requirements, as far as possible.

U.S. Pat. No. 5,107,830 (1992 Apr. 28; Younes) discloses a lung ventilator device. Ventilation to a patient is provided in response to patient effort. The free flow of gas from a piston, or similar air source, in response to patient inhalation is detected, the instantaneous rate and volume of flow are measured and the measurements are used as control signals to a drive motor for the piston to move the piston to generate a pressure which is proportional to the sum of measured and suitably amplified rate and volume of flow signals. Since the command signal to the pressure generator only changes subsequent to, and not in advance of, a change in flow and volume, the ventilator is subservient to the patient and provides a proportional assist to patient ongoing breathing effort during inspiration (Proportional Assist Ventilation, PAV).

US 20100116270 (2010 May 13; Edwards et al.) discloses Medical Ventilator System and Method Using Oxygen Concentrators. A medical ventilator system that allows the use of pulse flow of oxygen to gain higher FIO2 values and/or conserve oxygen is described. In one embodiment, the ventilator system includes an oxygen concentrator, a medical ventilator and a breathing circuit between the ventilator and a patient. In one embodiment, the oxygen concentrator includes a controller module that is configured to generate a trigger signal to initiate the distribution of one or more pulses of oxygen from the oxygen concentrator to the patient circuit at the onset of a ventilator supplied breath. A small flow of oxygen can be added in between pulses to aid in gaining higher FIO2.

### SUMMARY

It is an object of the invention to provide a medical ventilator (“ventilator”) that is substantially entirely mechanical. More particularly, it is desirable to avoid complicated electronics and associated mechanisms to operate the ventilator, while ensuring proper volume movement of air in-and-out of the lungs.

According to the invention, generally, a medical ventilator may comprise two pistons moving in unison with one another within respective two (a “first” and a “second”) cylinders. During an exhale phase of a breathing cycle, the first cylinder receives pressurized air which causes both pistons to move (upward). In the second cylinder, this creates a negative pressure to extract exhaled air from a patient's lungs. During an inhale phase of the breathing cycle, a weight acting on a link between the two pistons causes both pistons to move in an opposite (downward) direction, whereupon (i) the first piston delivers the pressurized air to the patient and (ii) the second piston vents the exhaled air.

The second cylinder may have a larger bore than the first cylinder. Alternatively, the piston in the second cylinder may have a longer stroke than the piston in the first cylinder. Alternatively, the second (exhale) cylinder may comprise two or more (a bank of) smaller cylinders, such multiple ones of the cylinder (such as a syringe) used for the first (inhale) cylinder.

Various configurations of cylinders, such as side-by-side or inline with one another are disclosed. Alternatively, rather than the pistons moving in unison with one another (and the cylinders being “fixed”, the cylinders may be moved in unison with one another while the pistons remain fixed.

According to an embodiment (example) of the invention, a ventilator may comprise: a first (inhale) cylinder having a first piston and a first connecting rod; a second (exhale) cylinder having a second piston and a second connecting rod; and a link between the two connecting rods so that the two pistons move in unison with one another. A weight may be disposed on the link to impart a downward force to the first and second pistons. Stops associated with the link may establish a desired Tidal Volume.

According to an embodiment (example) of the invention, a pneumatic cylinder medical ventilator system may comprise: a source of pressurized air comprising either (i) a first supply of air (or oxygen), such as may be supplied in a hospital room, such as at a pressure of 50-55 psi, or (ii) a second supply of air (or oxygen), such as may be supplied in a stand-alone tank, such as at a pressure of 200 psi (regulator included). The system may further comprise a first regulator valve, receiving the pressurized air; and a second regulator valve, controlling flow of air out of the first cylinder. A patient circuit may be selectively connected to (i) the inlet gas port of the second cylinder (FIG. 2A) and (ii) the outlet gas port of the first cylinder (FIG. 2B).

According to an embodiment (example) of the invention, a method of ventilating a patient ma comprise: with the dual cylinder medical ventilator system described herein, alternately providing (i) pressurized air to the patient for inhalation and (ii) “negative pressure” to the patient for assisting in removing exhaled air from the patient.

Generally, the ventilator disclosed herein comprises two pistons moving in unison with one another. The pistons are disposed in corresponding two cylinders. One cylinder may have a larger bore than the other. (Alternatively, one cylinder may have a longer stroke than the other.) A valve is associated with each cylinder.

In the overall system, a source of pressurized air is provided, and a patient circuit (tubing, mask) is provided.

In the main hereinafter, the actions and effects created by two cylinders are described, with one having a smaller bore than the other. These two cylinders may be referred to as the “small cylinder” and the “large cylinder”. The large cylinder may comprise a bank of two or more smaller cylinders, such as a plurality of the aforementioned small cylinders.

As mentioned, the two pistons may move in unison with one another. This may be effected by a mechanical linkage (link) between connecting rods associated with the pistons.

The small cylinder is arranged to receive pressurized air, and also to provide pressurized air to a patient via the patient circuit.

The large cylinder is arranged to receive air exhaled by the patient, and also to vent that air to the outside (environment).

During an exhalation phase of a breathing cycle, pressurized air is delivered to the small cylinder, its piston moves (rises) and the piston in the large cylinder also rises and creates a negative pressure for gathering (harvesting) exhaled air from the patient.

During an inhalation phase of the breathing cycle, pressurized air in the small cylinder is delivered to the patient, and previously exhaled air is vented from the large cylinder to the environment.

The valves associated with the cylinders are arranged such that they switch between the phases of the breathing cycle when end stops are reached. One end stop may be “fixed”, representing Tidal Volume=0. The other end stop may be movable (adjustable) to be set at a desired tidal Volume (such as a few hundred milliliters).

The ventilator disclosed herein may be made at least largely of plastic, such as PVC. Thus, it may be made inexpensively, and may be very suitable for one-time use. Some “ancillary” components such as valves and flow regulators and gauges may be reusable.

The invention is described more specifically in the description that follows.

Other objects, features and advantages of the invention(s) disclosed herein may become apparent in light of the following illustrations and descriptions thereof.

### BRIEF DESCRIPTION OF THE DRAWINGS

Reference will be made in detail to embodiments of the disclosure, non-limiting examples of which may be illustrated in the accompanying drawing figures (FIGs). The figures may generally be in the form of diagrams. Some elements in the figures may be stylized, simplified or exaggerated, others may be omitted, for illustrative clarity.

Although the invention is generally described in the context of various exemplary embodiments, it should be understood that it is not intended to limit the invention to these particular embodiments, and individual features of various embodiments may be combined with one another. Any text (legends, notes, reference numerals and the like) appearing on the drawings are incorporated by reference herein.

FIG. 1 is a diagram of a single acting pneumatic cylinder, illustrating some basic principles which may be applied to the invention.

FIG. 2 is a diagram of a two pneumatic cylinder approach, forming a basis of the invention.

FIG. 2A is a diagram illustrating the operation of a two pneumatic cylinder ventilator, in the exhale cycle, according to an embodiment of the invention.

FIG. 2B is a diagram illustrating the operation of a two pneumatic cylinder ventilator, in the inhale cycle, according to an embodiment of the invention.

FIGS. 3A and 3B show some variations in arranging the two cylinders, according to an embodiment of the invention.

### REFERENCE NUMERALS (SOME)

100 cylinders (100i=inhale, 100e=exhale)
104 pistons (104i=inhale, 104e=exhale)
106 vent ports (106i=inhale, 106e=exhale)
110 connecting rods (110i=inhale, 110e=exhale)
120 inlet gas ports (120i=inhale, 120e=exhale)
122 outlet gas ports (122i=inhale, 122e=exhale)
200 ventilator (system)
210 oxygen/air supply
212 oxygen/air supply
220 link (arm)
230 mass (weight)
310 connecting rod

There are some additional illustrations in the Appendices (1, 2, 3) provided herewith.

### DESCRIPTION

Various embodiments (or examples) may be described to illustrate teachings of the invention(s), and should be construed as illustrative rather than limiting. It should be understood that it is not intended to limit the invention(s) to these particular embodiments. It should be understood that some individual features of various embodiments may be combined in different ways than shown, with one another. Reference herein to “one embodiment”, “an embodiment”, or similar formulations, may mean that a particular feature, structure, operation, or characteristic described in connection with the embodiment is included in at least one embodiment of the present invention. Some embodiments may not be explicitly designated as such (“an embodiment”).

One of the difficulties in ventilation is providing the proper volume movement of air in-and-out of the lungs. If too much air is driven in with less being withdrawn, the patient may hyperventilate as CO2 concentration in the lungs accumulate. Conversely, withdrawing more gas than injecting causes oxygen deprivation. Both situation lead to patient discomfort, anxiety, and panic.

The invention disclosed herein provides a simple method for ensuring that equal volumes of air are injected and withdrawn for each breath. This may seem to be a simple task, but it is not. The reason this task is challenging is that although the same volume of air is required to inflate and deflate the lungs (i.e., no net inflation or deflation is a series of inflate/deflate cycles), the injection (inflation, inhale) portion (phase) of the cycle requires higher pressure in order to inflate the lungs, while lower pressure (vacuum) is required to deflate (exhale) the lungs. Therefore, the air being injected is compressed to provide the required gas flow inward. The gas in the lungs is fully expanded and must be withdrawn (exhaled). Hence, the volume of air under pressure is different than the expanded air volume in the lungs.

The invention disclosed herein utilizes a set of ‘single-acting pneumatic cylinder’ type devices as replacement for (instead of) traditional inflating and deflating bellows.

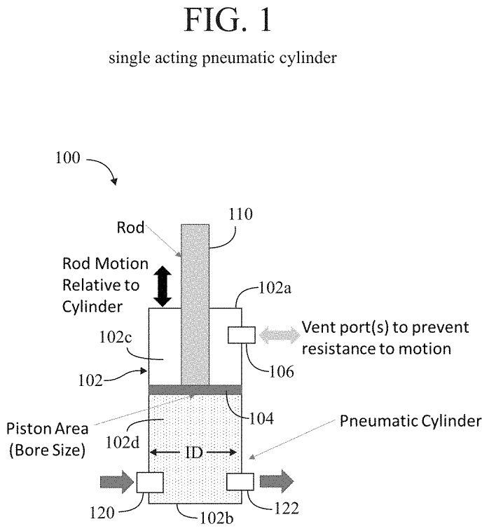

FIG. 1 illustrates an example of a one single acting pneumatic cylinder apparatus 100, a pair of which may be adapted for use with the present invention.

The apparatus 100 generally comprises:
a pneumatic cylinder 102 having a wall, an inside diameter (ID), and sealed at both of its top (upper) 102a and bottom (lower) 102b ends. Alternatively, the top of the cylinder may be open (not sealed);
a piston 104 capable of moving up and down (as illustrated) within the cylinder;
a connecting rod 110 extending from outside the cylinder, through the top end thereof, and capable of moving the piston up and down (axially) within the cylinder in response to a motive force provided by an external instrumentality (not shown).

An upper portion 102c of the cylinder may be defined as the area (i.e., volume) within the cylinder, above the piston 104.

A lower portion 102d of the cylinder may be defined as the area (i.e., volume) within the cylinder, below the piston 104.

The piston may comprise a simple, flat, disc-like structure having an outer diameter (OD) corresponding with an inside diameter (ID) of the cylinder. The piston may be provided with sealing means for creating a substantially air-tight seal between the piston and cylinder so that the piston can move—i.e., compress or decompress—air within the cylinder in the upper and lower portions thereof. The sealing means may simply comprise a reasonably precise fit between the OD of the piston and the ID of the cylinder (i.e., no separate pieces). Or, separate means such as O-rings may be provided around the circumference of the piston to effect the air-tight seal.

In response to the connecting rod moving up and down, the piston moves up and down. And, there may be mechanical stops incorporated into the cylinder to limit the piston's up and down movement (aka “stroke”).

One or more vent ports 106 may be provided in the wall (alternatively, in the top end) of the cylinder, above the piston to prevent resistance to piston movement (i.e., due to the air resisting being compressed). The vent ports may be disposed above a topmost (uppermost) position of the piston.

An inlet gas port 120 may be provided in the wall (alternatively, in the bottom end) of the cylinder, below a bottommost (lowermost) position of the piston. Similarly, an outlet gas port 122 may be provided in the wall (alternatively, in the bottom end) of the cylinder, below the bottommost (lowermost) position of the piston. The outlet gas port 122 may be integral with the inlet gas port 120 so that a single port may function as both the inlet and outlet gas ports. They are shown separately in some figures for descriptive purposes.

According to an aspect of the invention, two of these ‘pneumatic cylinder’ type devices (100) may be paired, using the ideal gas law. One of these devices (100) may be used to implement an “inhale phase” (i) of a breathing cycle, and another of these devices (100) may be used to implement an “exhale phase” (e) of the breathing cycle—a single breathing cycle being defined as one inhalation followed by one exhalation (or vice-versa). (The inhale phase of a breathing cycle may be referred to as the “inhale cycle”, and the exhale phase of the breathing cycle may be referred to as the “exhale cycle”.)

The ideal gas law, also called the general gas equation, is the equation of state of a hypothetical ideal gas. It is a good approximation of the behavior of many gases under many conditions, although it has several limitations. It was first stated by Emile Clapeyron in 1834 as a combination of the empirical Boyle's law, Charles's law, Avogadro's law, and Gay-Lussac's law. The ideal gas law is often written in an empirical form:

PV=nRT

where P, V, and T are the pressure, volume and temperature; n is the amount of substance; and R is the ideal gas constant. The PᵢVᵢ of the ‘Inhale (i) cycle’ and PₑVₑ of the ‘Exhale (e) cycle’ can be described as

PᵢVᵢ=nᵢRᵢTᵢ

PₑVₑ=nₑRₑTₑ

Since the same amount of substance/gas is injected and withdrawn nₑ=nᵢ; since the gas is the same Rᵢ=Rₑ; and temperature may be assumed to be the same for each cycle Tᵢ=Tₑ. (The difference between Ti and Te is negligible.) Therefore,

PᵢVᵢ=PₑVₑ

Vi and Ve represent the volumes of two cylinders. Vi represents the volume of gas to be injected into the lungs (inhale phase) and Ve represent the volume of gas extracted from the lungs. Post exhale, the pressure of the air withdrawn from the lungs is at atmospheric pressure (14.696 psi, at sea level). The volumes and pressures which would provide equal volume of gas for each cycle (i.e., for the inhale and exhale phases of the overall breathing cycle) should be identified.

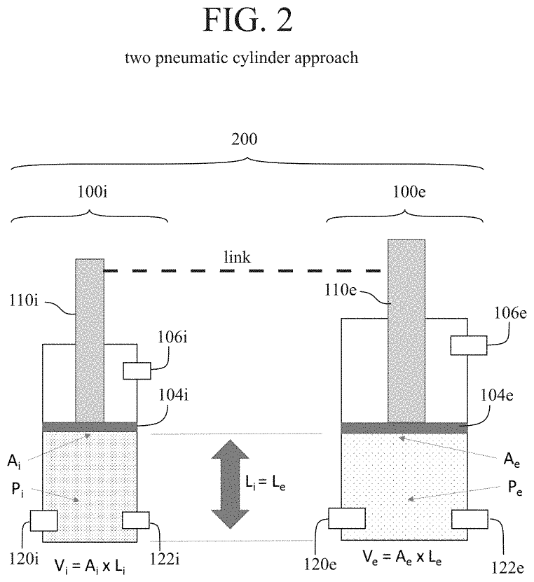

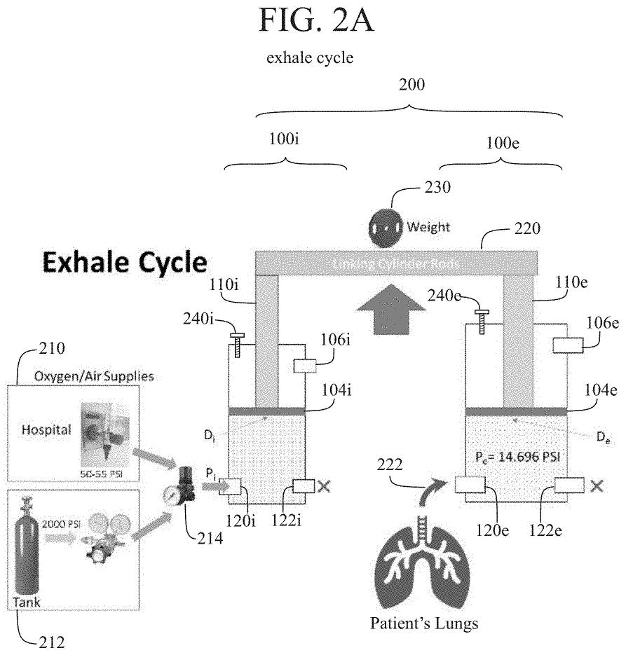

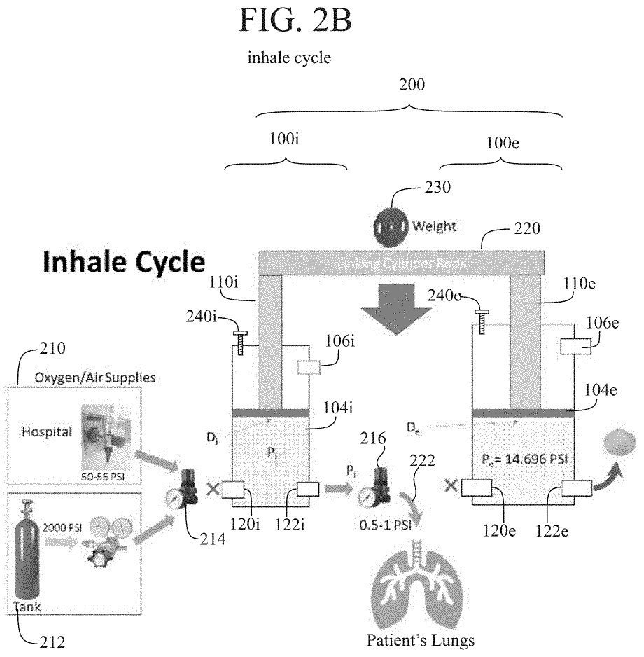

FIG. 2 shows two pneumatic cylinders (apparatuses), each of which may be of the type described with respect to FIG. 1—namely, one cylinder 100i may be used for the inhale (i) phase of a breathing cycle, and the other cylinder 100e may be used for the exhale (e) phase of a breathing cycle.

Generally, elements of FIG. 1, such as the cylinder 100 may be used for each of the two cylinders 100i and 100e disclosed and described with respect to FIGS. 2A, 2B, et seq., and other elements may also have “i” and “e” suffixes to indicate which of the two (or “dual”) cylinders they are associated with. For example piston 104 becomes piston 104i in cylinder 100i, and piston 104 becomes piston 104e in cylinder 100e.

The cylinder 100i may be designated as the “first” cylinder, and the cylinder 100e may be designated as the “second” cylinder. The first cylinder 100i may have a smaller diameter than the second cylinder 100e. For example, the diameter (Di) of the first cylinder 100i may be 1.5″ (3.75 cm), and the diameter (De) of the second cylinder 100e may be 3″ (7.5 cm).

For a typical round cylinder, the volume may be calculated by the stroke length (L) times the area (A) of the cylinder bore (piston), i.e. V=LA

PᵢLᵢAᵢ=PₑLₑAₑ

If Lᵢ=Le, then . . .

PᵢAᵢ=PₑAₑ

Assuming a round (and flat-top) piston, surface area A=πr²=π(D/2)², where r=piston radius and D=piston diameter. Therefore, Pi in terms piston diameter,

$$Pi = \frac{PeAe}{A i}$$

$$Pi = Pe \frac{π {r}_{e}^{2}}{π {r}_{i}^{2}} = Pe \frac{{D}_{e}^{2}}{{D}_{i}^{2}}$$

Thus, given that Pe is a constant, this characteristic expression represents combinations of two cylinder diameters where one cylinder (i=inhale cycle) would deliver a preset volume of pressurized air while the second would withdraw (e=exhale cycle) the same volume of gas at 1 atmosphere, for any equal stroke length. The diameter (volume) of the cylinder holding the pressurized gas will always be smaller than the cylinder withdrawing. The stroke length or rod travel distance represents a set Tidal Volume.

If the rods of these two cylinders are linked (connected) with one another (see dashed line “link” in FIG. 2), the exhale and inhale cycles (phases) may be generated as follows, referring to FIGS. 2A and 2B, respectively.

It bears mention that the illustrative breathing cycle described herein may be presented as (i) and exhale phase followed by (ii) an inhale phase. This is a matter of convenience for describing the principles involved, it being understood that a give breathing cycle ordinarily may start with an inhale phase followed by an exhale phase, and so on.

FIG. 2A shows an apparatus (or system) 200, having two cylinders 100i and 100e, such as were described with respect to FIG. 2, performing an exhale phase of a breathing cycle. FIG. 2B shows the apparatus (or system) 200 performing an inhale phase of a breathing cycle.

FIG. 2A (exhale phase) also shows:
a supply 210 of air (or oxygen), such as may be supplied in a hospital room, such as at a pressure of 50-55 psi
alternatively, or additionally, another supply 212 of air (or oxygen), such as may be supplied in a stand-alone tank, such as at a pressure of 2000 psi (regulator included)
a pressure regulator 214, receives pressurized air (or oxygen) from the one or the other of the supplies 210 and 212, and provides the air (or oxygen) to the inlet gas port (120) of the cylinder 100i.
The outlet gas port 122 of the cylinder 100i is closed, as indicated by “X”.
The outlet gas port 122 of the cylinder 100e is also shown as being closed, as indicated by “X”.

With regard to the pressure regulator 214 shown in FIGS. 2A and 2B, and associated with the inlet port of the cylinder 100i, it should be understood that in conjunction with the pressure regulator 214, such as between the pressure regulator and the cylinder 100i, there should be a flow valve to control inflation rate, as is indicated in the illustrations in the Appendix 1 at Pages 1 and 2.

Similarly, with regard to the pressure regulator 216 shown in FIG. 2B, discussed below and associated with the outlet port of the cylinder 100i, it should be understood that in conjunction with the pressure regulator 216, such as between the pressure regulator and the cylinder 100i, there should be a flow valve to control inflation rate, as is indicated in the illustrations in the Appendix 1 at Pages 3 and 4.

It is important to control both pressure and flow. Two separate elements may be used, as discussed above, or a combined pressure/flow regulator may be used for the elements 214 and 216.

Recall that the inlet gas port (120) and outlet gas port (122) for a given cylinder may be a single port, not the two ports that are illustrated, two separate (inlet and outlet) ports being shown separately in the diagrams to illustrate their function at a given phase of the overall breathing cycle. Having a single port in a cylinder performing two (inlet and outlet) functions is clearly shown in Appendix 1 which shows each cylinder as having only a single port and a valve for controlling the desired function of the respective cylinder and port at a given phase of the breathing cycle.

FIG. 2A (exhale phase) also shows:
a patient's lungs connected (in fluid communication with)—via appropriate conventional tubing, mask, etc. (222 not shown in any detail, aka “patient circuit”)—to the inlet gas port 120e of the cylinder 100e.

FIG. 2B (inhale phase) shows many of the elements of FIG. 2A, and also shows:
patient's lungs connected (in fluid communication with)—via the appropriate conventional tubing, mask, etc. (222)—to the outlet gas port (120) of the cylinder 100i, via a pressure regulator 216. This regulator 216 controls the flow of air from (out of) the cylinder 100i.
The inlet gas port 120i of the cylinder 100i is closed, as indicated by “X”.
The inlet gas port 120e of the cylinder 100e is also shown as being closed, as indicated by “X”.

FIGS. 2A and 2B also show a mechanical link (or arm) 220 (compare “link” in FIG. 2) connecting the rods (110) of the two cylinders 100i and 100e together so that they operate in unison with one another. Typically, the pistons (104) in the two cylinders (100) may move up and down the same amount as one another, and simultaneously with one another.

A weight (mass) 230 may be disposed on the link 220 to provide a constant downward (restoring) force on the arm 220, hence on the pistons (104) of the two cylinders. A typical mass for the weight may be 35 pounds (16 kg). In lieu of a weight, a spring (air or mechanical) could be incorporated into the design to provide a similar downward force on the two pistons.

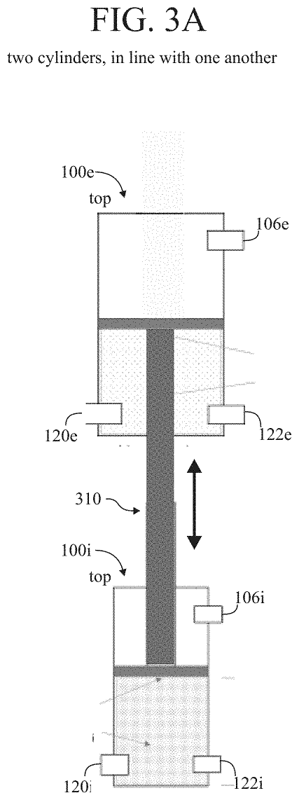

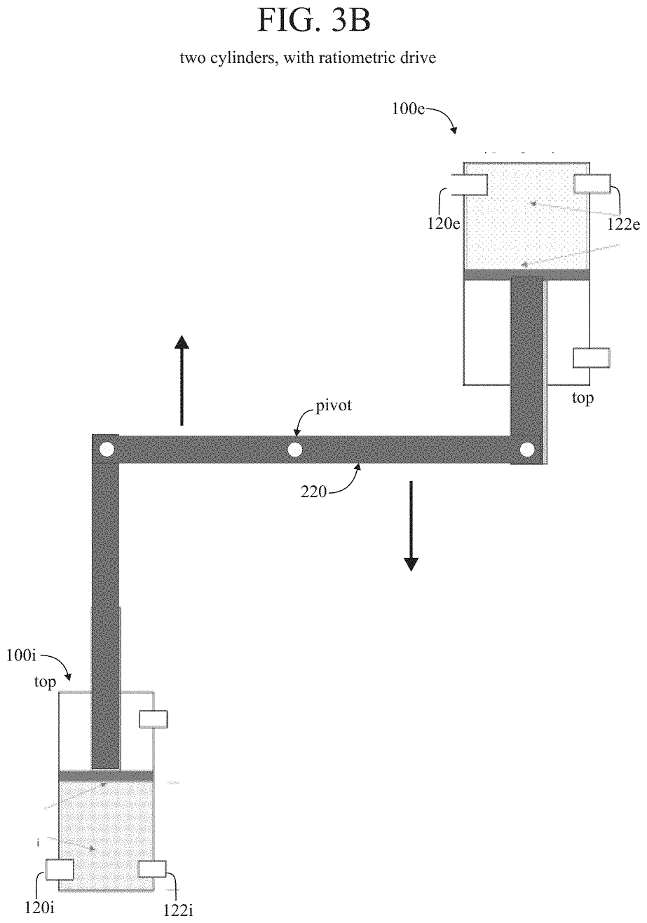

FIG. 3A shows that, alternatively, the second cylinder 110e could be disposed vertically above the first cylinder 110i. The two cylinders could share the same connecting rod 310 (compare 110). The rod could enter the top of the first cylinder 100i (as described before), and enter the bottom of the second cylinder 100e (contrary to before, where a rod also entered the top of the second cylinder. A separate link (220) is not shown.

Alternatively, the two cylinders could be oriented horizontally, to the left and right of each other. In either case, the two cylinders would be in line (e.g., coaxial with) one another. A weight (230) is not shown, other means such as springs may be provided for providing the restoring force on the pistons.

The size (particularly the bore) of the two cylinders may be different than one another, in which case the stroke (axial movement) of the two pistons may be the same as one another, but the resulting volume of air moved by the two cylinders would be different than one another. Also, note that when the “rod side” volume of the cylinder is utilized (such as in the cylinder 100e), the rod area is subtracted from the piston area to determine the net piston area used in calculating volume.

FIG. 3B shows that appropriate linkage could be incorporated into the link 220 so that the piston of one cylinder can be caused to move a different “ratiometric” amount than the piston of the other cylinder. For example, the link (220) could have a pivot located near its middle (such as halfway between the two cylinders), and one of the cylinders could be inverted with respect to the other, to achieve a desired ratio. Gears and the like could also be incorporated to achieve the desired ratio. The result is that one cylinder may have a longer stroke than the other cylinder. This may allow for the two cylinders to have the same bore (ID) as one another, while achieving a similar result that is obtained by having one cylinder (100e) having a larger bore than the other cylinder (100i).

### Operation of the System

A patient's overall breathing cycle may comprise an exhaust phase (or exhaust cycle) and an inhale phase (or inhale cycle). Or vice-versa. A methodology for ventilating a patient will now be described, with respect to FIGS. 2A, 2B.

### Exhale Cycle

Referring to FIG. 2A, during an exhale cycle, pressurized gas (pressure=Pi) is provided from one or the other of the pressure sources 210/212 into the smaller diameter cylinder 100i. The gas pressure (Pi) causes the piston 104i in the cylinder 100i to urge (push, move) the rod 110i upward which, via the ink 220 will pull the piston 104e in the cylinder 100e upward via its connecting rod 110e.

As the piston 104e in the cylinder 100e moves upward, a negative pressure (‘vacuum’) is created in the lower portion (102d) of the cylinder 100e. This pressure will be lower than atmospheric pressure (Pe=14.696 PSI) thereby withdrawing (sucking, extracting) gas from the patient's lungs.

The gas pressure (Pi) times the surface area of the piston 104i in the cylinder 100i generates an upward (as viewed) force which will move the link 220, pistons 104i, 104e and weight 230 upward. (This may be referred to as the “upward cycle”.) Gas flow (Pi) from the pressurized gas sources 210 and/or 212 may be terminated when the pistons 104i and 104e reach the top of their stroke. The length of the upward stroke (in conjunction with piston surface areas) may be adjusted to establish a given, desired Tidal Volume (TV).

Upward motion of the pistons may be limited by a simple adjustable mechanical stop 240i or 240e incorporated into either one or both of the cylinders 100i, 100e, respectively. The stops 240i and 240e may simply be set screws extending through the top ends of the respective cylinders 100i and 100e. Alternatively, and perhaps more preferably, stops (shown and described hereinbelow in the Appendix 1 document) for limiting both upward and downward motion of the pistons may be positioned to act upon and limit movement (up and down) of the arm 220 connected to the two connecting rods 110i and 110e.

During the “exhale cycle” represented in FIG. 2A, the inlet gas port 120i of the cylinder 100i is open, the outlet gas port 122i is closed (X). Recall (from above) that the outlet gas port 122 may be integral with the inlet gas port 120 so that a single port may function as both the inlet and outlet gas ports. If the two ports are realized separately, the exhaust port would need to be closed, such as with a solenoid.

Also, during the exhale cycle, the inlet gas port 120e of the cylinder 100e is open, to pull air from the patient's lungs 220, and the outlet gas port 122e is closed (X).

### Inhale Cycle

With reference to FIG. 2B, the inhale cycle (i.e., inhale phase of the overall breathing cycle) commences, with or without a delay (Td), after completion of the exhaust cycle.

It bears mention that the order (sequence) of the inhale and exhale cycles may, of course, be reversed, depending on one's point of view. However, the breathing cycle is appropriately presented as exhale first, then inhale, due to the exhale cycle being initiated first, upon the application of pressurized gas (Pi) to the cylinder 100i, as discussed above with respect to FIG. 2A—hence the sequence of exhale/inhale/exhale/inhale, etc, described herein.

During the inhale cycle, delivery of pressurized gas (Pi) to the cylinder 100i stops (ceases), if it has not already been turned off, and pressurized gas which was “stored” (accumulated) in the cylinder 100i flows out of the pressurized cylinder 100i, through the exhaust gas port 122i, and though a pressure regulator 216 into the patient's lungs. (Appendix 1 shows the switching valves that control the flow into the breathing circuitry to switch between the inhale and exhale cycles.)

The weight 230 on top of the link 220 utilizes gravity to keep the piston 104i moving downward to maintain pressure in the cylinder 100i. The piston 104e will also move downward, since it is linked to the piston 104i. (This may be referred to as the “downward cycle”.) During this phase,
the inlet port 120i is closed (X)
the outlet port 122i is open, delivering gas (air) to the patient
the inlet port 120e may be closed (X)
the outlet port 122e may be open, and as the piston 104e moves downward, the patient's previously expelled gas in the cylinder 100e may be expelled into the atmosphere (such as through a suitable filter to remove contaminants, or into or a suitable container).

In lieu of a weight, an arrangement of springs and the like may be used to maintain the desired downward force on the pistons. The force need not be constant, rather it may have a “profile” (force v distance).

### The Cycle(s), Tidal Volumes, and Some Other “Details”

Using the dual cylinder medical ventilator system described above, a method of ventilating a patient may comprise alternately providing pressurized air to the patient for inhalation and assisting in removing exhaled air from the patient. Or vice-versa (exhale, then inhale).

The Exhale Cycle may resume (with or without a designated delay) once the downward stroke of the Inhale Cycle is completed, and both cylinders are empty. The downward stroke length may be preset with a movable stop, similar to the upward stroke stop 240, but in the bottom end of the cylinder(s), that can be adjusted to accommodate the patient's Tidal Volume.

In the examples set forth herein, the following “standard” exemplary cylinder bores may be utilized.
If, for example, a 3″ (7.5 cm) bore (ID) cylinder is selected for the (larger diameter) “exhale” cylinder 100e then the piston area is 7.068 in². If an 800 ml (48.819 in³) tidal volume is required, then the stroke length could be set to 6.91″ (48.819 in³/7.068 in²).
If, for example, a 1.5″ (3.75 cm) bore (ID) cylinder is selected for the (smaller diameter) “inhale” cylinder 100i, then the piston area is 1.767 in².

The results indicate that when the input pressure (Pi) is equal to 58.784 PSI (or 44 PSIG) utilizing the 3″ and 1.5″ bore diameter cylinder set, then any linear stroke when the rods are connected will guarantee that the Tidal Volume to be equal in the inhale and exhale cycles.
PSI (pounds per square inch) refers to the amount of pressure (force) exerted on an object having a surface area of one square inch. PSIG (pounds per square inch, gauge) is a unit of pressure relative to the surrounding atmospheric pressure, and the “G” in PSIG means that it is a relative measurement. (Atmospheric pressure is ˜14.7 psi. Hence 58.7 psi would correspond with 44 psig.)

Furthermore, when 44 PSIG is delivered to the 1.5″ diameter cylinder 100i, it is able to lift a total maximum weight of 77.9 lbs (44 psi×1.767 in²). This lifting force will act upon the piston 104i, the rod 110i, the link 220, the rod 110e and the piston 104e, plus the added weight 220, plus frictional forces in the system. The net weight on the downward cycle (FIG. 2B) translates to pressure in the small cylinder that is able to maintain sufficient flow (inhale) into the lungs.

In this example, ideal or custom diameter cylinders are selected. Again, if a 3″ bore cylinder is selected for the exhale cylinder 1002, then the piston area is 7.068 in². If an 800 ml (48.819 in³) tidal volume is required, then the stroke length could be set to 6.91″ (48.819 in³/7.068 in²). However, knowing that 50 PSIG is available at the hospital connection, it may be advantageous to optimize the diameter (ID) of the smaller inhale cylinder 100i.

Using the characteristic equation by solving for Di,

$$Di = \sqrt{\frac{{PeDe}^{2}}{Pi}} = \sqrt{\frac{14.696 * {3}^{2}}{50 + 14.696}} = {1.43}^{″}$$

Therefore, because the pressure (Pi) supplied to the system 200 is larger than in the previous example, for the same stroke length, the diameter may be decreased slightly to reflect the reduced volume required. However, at 50 PSIG and a diameter of 1.43″ (piston area ˜1.6 in²) the upward force is 80 lbs.

There has thus been shown and described, a dual (two) cylinder medical ventilator, system and method that is simple, mechanical (no electronics required), very portable, inexpensive, effective and easy to operate.

### APPENDIX 1

Appended hereto and forming a portion of the disclosure hereof is a document entitled “Control Methodology”, 5 pages, which shows in greater detail the operation of the two piston ventilator disclosed herein.

Some elements (components) which may not have been mentioned above (FIGS. 2, 2A, 2B), or which were only briefly and broadly described, may be shown in greater detail in this document. For example,
a fixed end stop disposed below the link (220) to limit downward motion of the pistons, at Tidal Volume=0
an adjustable (movable) end stop disposed above the link (220) to limit upward motion of the pistons, at a desired Tidal Volume, such as up to 200 ml, 400 ml, 500 ml, or 600 ml. (This replaces the end stops 240i and 240e described hereinabove.)

### Page 1 (Exhalation Initiation)

This page shows:
a first (left, as viewed) cylinder corresponding with the previously described cylinder 100i
a second (right, as viewed) cylinder corresponding with the previously described cylinder 100e
the left cylinder may have a smaller diameter than the right cylinder, as described hereinabove
a 50 psi source (compare 210, 212) of pressurized air
a flow regulator (compare 214)
a link (compare 220) disposed between the two cylinders to cause their pistons (104) to move up and down in unison with one another
a weight (compare 230) exerting a downward force on the link, such as described hereinabove
a fixed end stop disposed below the link to limit downward motion of the link and associated pistons
a movable end stop disposed above the link to limit upward motion of the link and associated pistons
a Tidal Volume Scale is shown (in hundreds of milliliters) which, in conjunction the with moveable end stop may be used to set the desired Tidal Volume for a given patient.
a first valve, such as a spool valve, is associated with the left cylinder, and is by design balanced under pressure and therefore remains “open” or “closed” depending on the spool's mechanical position
a second valve such as a spool valve, is associated with the right cylinder, and is by design balanced under pressure and therefore remains “open” or “closed” depending on the spool's mechanical position
these two valves were not shown in the FIG. 2 descriptions of the apparatus
each valve is shown twice in the diagram, once positioned near the respective cylinder with which it is associated to explain the air flow in or out of the cylinder, and once on the link to illustrate how the valve may be operated (open or closed) depending upon the position of the link.
For example, in use, as pressurized air is being delivered to the first cylinder (100i), the two valves disposed on the link will bump against the fixed end stop at Tidal Volume Zero to initiate pressurized gas flow to the small cylinder (100i), initiating its upward movement, thereby pulling the large cylinder (100e) upward and generating a negative pressure to remove the CO2 (oxygen depleted air) from the lungs
Flow of pressurized air is shown (curved arrow) into the inlet port 120i of the cylinder 100i
Flow of air from the patient's lungs is shown (curved arrow) into the inlet port 120e of the cylinder 100e

It bears mention here that the first part (phase) of the breathing cycle likely to be initiated by the apparatus would be the exhale phase. This may be attributed to a “start” situation, before pressure is provided, where the two pistons fall to the bottom of their travel, due to the weight (230) and air escaping into the environment. When pressure is applied, the pistons start to move upward, and the exhalation phase (pages 1, 2 of the Appendix) starts. At the end of the exhalation phase, the valves contact the movable end stop (set at the desired Tidal Volume), and the inhalation phase (pages 3, 4 of the Appendix) of the breathing cycle begins as the pistons commence their downward travel. Hence, the descriptions of the breathing cycle set forth herein being “exhale, then inhale” (rather than “inhale, then exhale”).

### Page 2 (Exhalation Step)

This page shows the same elements (components) that were shown on Page 1, and illustrates what happens during the exhalation phase of the breathing cycle.

A duration of the Exhalation Step (compare FIG. 2A) may be set (by the physician) by setting the flow regulator (214) to adjust the fill rate of the small cylinder (100i).

The images in Appendix 1 are more detailed in some respects than in FIGS. 2A/B, and include a flow regulator which is separate from the pressure regulator (214) and is set to a fixed pressure due to cylinder size selection. A flow regulator controls the inflation rate of the cylinder and therefore the exhale duration cycle. Also a separate flow regulator may be disposed in the circuit after the pressure regulator (216).

At a common “default” setting of 12 breaths per minute (bpm), the duration of a breathing cycle is about 5 seconds. Exhalation is about 1-2 seconds of the 5 second duration. Inhalation is about 3-4 seconds of the breathing cycle.

The illustration on Page 2 shows that the link (220) has moved upward towards the movable end stop to a position corresponding with approximately 400 ml Tidal Volume.

This represents the end of the exhalation phase of the breathing cycle.

### Page 3 (Inhalation Initiation)

This page shows the same elements (components) that were shown on Page 1.

This page additionally shows
a filter (P100)

The illustration on Page 3 shows that the rod (220) has (at the end of the exhalation phase) moved upward to the movable (adjustable) end stop to a position corresponding with approximately 450 ml Tidal Volume, whereupon
the link (and valves) stops
the valves are operated (switched);
the weight (230) starts to force the link (220) and pistons (104i, 104e) downward; and
the inhalation phase of the breathing cycle begins.

More particularly, when the link (220) and the valves reach the movable (adjustable) end stop, the following things happen:
the small cylinder (100i) valve switches over from (i) providing pressurized air from the source (210, 212) to the small cylinder (100i) to (ii) communicating air in the lower portion (102d) of the cylinder (100i) to the patients lungs, optionally through a flow regulator (as shown). The air pressure being delivered to the patient may be regulated to 0.5-1.0 psi. (One regulator controls the inlet gas pressure to the cylinder inflation pressure, another regulator limits the cylinder inflation pressure down to the lung inhalation pressure.
the large cylinder (100e) valve switches over from (i) communicating air from the patient's lungs to the bottom portion (102d) of the large cylinder (100e) to (ii) expelling air to the environment, optionally through a filter; and
the inhalation phase of the breathing cycle will commence, as illustrated at pages 3, 4 of the Appendix.

Before going further, it is worth noting that the following adjustments may be made (by the physician):
the desired Tidal Volume has been set by adjusting the movable end stop; and
the flow regulator that sets the inflation rate of the small cylinder at inlet 120i; and
the pressure regular (216) and flow regulator at output of 122i.

The illustration at page 3 of the Appendix shows:
flow (curved arrow) of previously pressurized air out of the outlet port (122i) of the small cylinder (100i)
flow (curved arrow) of air which was extracted from the patient's lungs, out of the outlet port (122e) of the cylinder (100e), through a filter (P100) and into the environment.

Recall that each cylinder 100i and 100e may have a single port acting as its inlet and outlet gas ports. In other words:
the inlet port 120i and the outlet port 122i of the cylinder 100i may be implemented as a single port
the inlet port 120e and the outlet port 122e of the cylinder 100e may be implemented as a single port

In this inhalation phase of the breathing cycle, the two valves bump against the Movable End Stop, and they both open, resulting in the following:
With the weight 230 acting upon the link 220, this causes the pistons 104i and 104e in the cylinders 100i and 100e, respectively, to move downward which (with respect to the cylinder 100i) allows for and initiates the release (arrow) of a volume of previously pressurized gas out of the cylinder 100i, through a flow regulator (216), into the patient's lungs.
The piston 104e in the cylinder 100e also moves downward, and CO2 gas (previously gathered exhaled, de-oxygenated air) in the large cylinder 100e is pushed out (arrow) and vented in the environment through a particulate filter (P100).

### Page 4 (Inhalation Step)

This page shows the same elements (components) that were shown on Page 1.

This page shows that the link (220) has begun to move downward, away from the movable end stop and towards the fixed end stop.

As mentioned before, at a common “default” setting of 12 breaths per minute, the duration of a breathing cycle is about 5 seconds. Exhalation is about 1-2 seconds of the 5 second duration. Inhalation is about 3-4 seconds of the breathing cycle.

The end of the inhalation phase of the breathing cycle is demarked by the link (220) reaching the fixed end stop, at which point the following things happen:
the small cylinder (100i) valve switches over from (ii) communicating air in the lower portion (102d) of the cylinder (100i) to the patients lungs to (i) providing pressurized air from the source (210, 212) to the small cylinder (100i)
the large cylinder (100e) valve switches over from (ii) expelling air to the environment, optionally through a filter to (i) communicating air from the patient's lungs to the bottom portion (102d) of the large cylinder (100e) to; and
the exhalation phase of the breathing cycle will commence, as illustrated at pages 1, 2 of the Appendix.

The duration of the Inhalation Step is set by the Tidal Volume, plus the pressure and flow regulators that reside on the tubing going into the lungs.

There has thus been described, in the text above as may be illustrated by the descriptions on pages 1-4 of the Appendix, an apparatus, system and method for assisting a patient to breathe by
(Pages 1-2) extracting used air from the patient's lungs during an Exhale Step (or exhale phase of an overall breathing cycle)
(Pages 3-4) providing pressurized air to the patient's lungs during an Inhale Step (or inhale phase of an overall breathing cycle)

Notably, the system may operate substantially entirely mechanically, without electronics.

The system basically comprises:
two pistons, a piston 104i in a smaller cylinder 100i and a piston 104e in a larger cylinder 100e;
a link 220 which will ensure that the two pistons move up and down in unison with one another;
a source of pressurized air;
two valves, one per cylinder; and
two flow regulators, one servicing the inlet port (exhalation phase; pages 1-2) and one servicing the outlet port (inhalation phase pages 3-4) of the pressurized cylinder.

A weight 230 is disposed on the link to provide a downward (restoring) force upon the pistons.

Mechanical stops establishing limits for upward and downward movement of the pistons, and setting the desired Tidal Volume.

The two valves were described and illustrated as being mounted on the link (220), which is moving up and down. Assuming (reasonably) that the pressurized air source, cylinders and patient circuit are “fixed” (not moving), some flexible tubing may be required between the valves and their respective cylinders. This may introduce a “weak link” into the system, the tubing being flexed incessantly and repeatedly, such as at 12 cycles per minute, or 17280 times per day. And, a single patient's time “on the ventilator” is typically measured in days (plural)! For 5 days of treatment, approximately one hundred thousand flexures of the tubing (and associated fittings) may occur. Although flexible tubing is regularly utilized for breathing air circuits and may be required to be changed between patients, it may be advantageous to eliminate the tube and fitting movements.

### Page 5

This page illustrates an alternative to moving the valves relative to the cylinders. In this arrangement, the valves remain in a fixed position—i.e., they do not need to move relative to the supply, the pistons, the patient circuit, etc.

In this “embodiment”, the Spool Valves are fixed with respect to the cylinders, rather than being mounted to the link (220).

The Tidal Volume Scale linkage may be adjustable with respect to the link (220). The valves may be bumped against the Cylinder Rods Linkage (defined as Tidal Volume=0 when cylinders are empty) to initiate pressurized gas flow to the small cylinder 100i. The Tidal Volume Setting based on the Tidal Volume Scale determines the distance the rods move upward. Once the Tidal Volume Scale bumps the valves, the desired Tidal Volume is reached. The Spool Valves reverse direction and downward movement is initiated.

### APPENDIX 2

Appended hereto and forming a portion of the disclosure hereof is a document entitled “Alternate Embodiment” which shows some alternate embodiments, as follows

Page 1 shows an alternate embodiment where weight (230) placement reduces (lowers) the center of gravity. Note that the connecting rods are disposed in the operative parts of the cylinder, below their respective pistons. Hence, as mentioned with respect to FIG. 3A (concerning the “inverted” cylinder 100e), when the rod side volume of the cylinder is utilized the rod area must be subtracted from the piston area when calculating net area and volume.

Note also that a complete ventilator apparatus is shown on the left-hand side of the illustration. Here, the size difference between the two cylinders is apparent. And the weights are shown disposed below the link.

Page 2 shows an alternate embodiment similar to that shown in FIG. 3A, with one cylinder disposed in-line with and above (or below) the other. A cylinder rod linkage (link 220) is shown “connecting” the two (or common single) connecting rods.

Page 3 shows an alternate embodiment similar to that shown in FIG. 3A, with one cylinder disposed in-line with and above (or below) the other. In contrast with the embodiment shown at Page 2 (and many of the other embodiments described herein, in the Page 3 embodiment, rather than the connecting rods(s) moving and being connected with one another by a link (and the cylinders are fixed), in this embodiment it is the cylinders that move and the connecting rods that are fixed. And, in this case, the link (compare 220) connects the two cylinders together (rather than connecting the rods together) to move in unison with one another.

Page 4 shows some alternate embodiments where a single or dual double-ended cylinders (these double-ended cylinders push one object while pulling another) simplify the mounting of weights to a coaxial design which simplifies mechanics; plus provides additional means for attaching the Tidal Volume adjustment.

### Some Final Comments

There has been described, hereinabove, a dual cylinder ventilator apparatus that is substantially entirely mechanical, including (i) the two cylinders, and (ii) the control mechanism (e.g., mechanical stops, etc.).

In the embodiment(s) described above, typically the small cylinder has a small connecting rod, and the large cylinder has a large connecting rod. Alternatively, the small cylinder could have a large connecting rod, and the large cylinder could have a small connecting rod.

In yet another embodiment (not shown), rather than having a single cylinder on each of the inhale and exhale sides, a plurality of cylinders may be used for either or both of the inhale and exhale sides to accommodate tight spaces or other restrictions as long as the sum of their respective areas (Ai and/or Ae) complies with the relationship Pi Ai=Pe Ae.

### APPENDIX 3

This shows an embodiment which may use disposable syringes as the cylinders. The Inhale cylinder may be a single syringe. The Exhaust cylinder may comprise a plurality (four shown) of syringes. In other words, a larger displacement cylinder (such as the exhaust cylinder) may have a larger bore (diameter), a larger stroke, or may comprise a bank of two or more smaller cylinders.

Appendix 3 illustrates what may be considered to be a disposable device, using one 300 cc syringe for inhale and four additional 300 cc syringes for exhale. The exhale syringes (bank of cylinders) may be disposed around the single inhale syringe (cylinder). The “numbers2 work out very well with supply pressure set to 44 psi (for the specific 300 cc syringes), but may change with another supplier's syringes as the piston size may be different. Ideally, the syringes, tubing, pressure and flow regulators, as well as the valves can all be made to be single use. The structure for holding the syringes and weights can be the only non-disposable items.

### Controls, Etc.

It is possible, and within the scope of the invention, to implement the controls with electronics, such as with appropriate sensors, solenoids, actuators, and the like. And, once the control mechanism is electronic, it can readily be computer-controlled. It is also possible to control the movement of the pistons with mechanical means such as actuators.

With the addition of another valve(s), pressurized air could be used to push down one (or both) of the pistons, instead of using a weight (or to reduce the mass of the weight). However, if running on oxygen only, then the gas is wasted to perform work that gravity can do. If that is not a consideration (such as if air, rather than oxygen is being used), then the weights can be eliminated.

The operation of the system may have been described with a sequence of exhale, inhale . . . (exhale first, rather than inhale first). Usually ventilators are powered up prior to connecting to the breathing circuit, so the cycle initiation is not critical. Due to the weights, our cycle starts with exhale, but if gas is used for the down cycle then we can start with inhale. In other words, using compressed gas (maybe a secondary cheap air compressor source so Oxygen is not wasted) as a compression force to replace (or augment) the weights.

Several ventilator designs may be found at:

https://www.businesswire.com/news/home/20200507005278/en/CoVent-19-Challenge-Attracts-200-Ventilator-Design-Submissions

(CoVent-19 Challenge Attracts More Than 200 Ventilator Design Submissions as 7 Teams Build Working Prototypes in Finalist Round)

Some general information about ventilators may be found at:

https://www.youtube.com/watch?v=7vLPefHYWpY

(A Guide To Designing Low-Cost Ventilators for COVID-19)

Here is a ‘pneumatic’ ventilator device:

https://www.youtube.com/watch?v=tfpG_ZUk1H0

(Helix Portable Ventilator—Adult or Paediatric)

See also:

https://www.youtube.com/watch?v=gk_Qf-JAL84

(Mechanical Ventilation Explained—Ventilator Settings & Modes (Respiratory Failure))

While the invention(s) may have been described with respect to a limited number of embodiments, these should not be construed as limitations on the scope of the invention(s), but rather as examples of some of the embodiments of the invention(s). Those skilled in the art may envision other possible variations, modifications, and implementations that are also within the scope of the invention(s), and claims, based on the disclosure(s) set forth herein.

What is claimed is:

1. Ventilator comprising:
a first cylinder having a first piston and a first connecting rod;
a second cylinder having a second piston and a second connecting rod; and
a link between the two connecting rods so that the two pistons move in unison with one another.

2. The ventilator of claim 1, further comprising:
an inlet port on the first cylinder for receiving pressurized gas from a source;
an outlet port for delivering pressurized gas to a patient circuit; and
an inlet port on the second cylinder for receiving air exhaled by a patient; and
an outlet port on the second cylinder for venting the exhaled air.

3. The ventilator of claim 1, further comprising:
a combined inlet/outlet port on the first cylinder for receiving pressurized gas from a source and for delivering pressurized gas to a patient circuit; and
a combined inlet/outlet port on the second cylinder for receiving air exhaled by a patient and for venting the exhaled air.

4. The ventilator of claim 1, further comprising:
a first valve associated with the first cylinder for switching between receiving pressurized gas from a source and delivering pressurized gas to a patient circuit; and
a second valve associate with the second cylinder for switching between receiving air exhaled by a patient and venting the exhaled air.

5. The ventilator of claim 1, further comprising:
a fixed stop (or reference point) at Tidal Volume=0; and
a movable end (or set point) to select a desired Tidal Volume.

6. The ventilator of claim 5, further comprising:
a first valve associated with the first cylinder for switching from receiving pressurized gas from a source to delivering pressurized gas to a patient circuit when a limit associated with a desired Tidal Volume is achieved; and
a second valve associate with the second cylinder for switching from receiving air exhaled by a patient to venting the exhaled air when a limit associated with zero Tidal volume is achieved.

7. The ventilator of claim 1, wherein:
the second cylinder comprises a bank of smaller cylinders (Appendix 3).

8. A method of ventilating a patient, comprising:
with the dual cylinder medical ventilator system of claim 1, alternately providing (i) pressurized air to the patient for inhalation and (ii) “negative pressure” to the patient for assisting in removing exhaled air from the patient.

9. Pneumatic cylinder medical ventilator, comprising:
a first pneumatic cylinder having a cylindrical wall, a first diameter (ID), a first (top) end, and a second (bottom) end;
a first piston disposed in the first cylinder and capable of moving up and down within the first cylinder;
a first connecting rod extending from outside the first cylinder, through the top end thereof, and capable of moving the first piston up and down (axially) within the first cylinder in response to a motive force provided by an external instrumentality;
wherein a first (upper) portion of the first cylinder is defined as an area (i.e., volume) within the first cylinder, above the first piston;
wherein a second (lower) portion of the first cylinder is defined as an area (i.e., volume) within the first cylinder, below the first piston;
a second pneumatic cylinder having a cylindrical wall, a second diameter (ID), a second (top) end, and a second (bottom) end;
a second piston disposed in the second cylinder and capable of moving up and down within the second cylinder;
a second connecting rod extending from outside the second cylinder, through the top end thereof, and capable of moving the second piston up and down (axially) within the second cylinder in response to a motive force provided by an external instrumentality;
wherein a second (upper) portion of the second cylinder is defined as an area (i.e., volume) within the second cylinder, above the second piston;
wherein a second (lower) portion of the second cylinder is defined as an area (i.e., volume) within the second cylinder, below the second piston; and
a link connecting the first and second connecting rods, causing the first and second pistons to move, in unison, when one or the other of the pistons is caused to move by an external force (such as pressurized air).

10. The pneumatic cylinder medical ventilator of claim 9, further comprising:
first vent ports disposed in the upper portion of the first cylinder, above the first piston to prevent resistance to piston movement (i.e., due to the air resisting being compressed);
second vent ports disposed in the upper portion of the second cylinder, above the second piston to prevent resistance to piston movement (i.e., due to the air resisting being compressed);
a first inlet gas port provided in the lower portion of the first cylinder (in the wall, alternatively, in the bottom end) of the first cylinder, below a bottommost (lowermost) position of the first piston;
a first outlet gas port, which may be integral with or separate from the first inlet gas port, provided in the lower portion of the first cylinder (in the wall, alternatively, in the bottom end) of the first cylinder, below a bottommost (lowermost) position of the first piston;
a second inlet gas port provided in the lower portion of the second cylinder (in the wall, alternatively, in the bottom end) of the second cylinder, below a bottommost (lowermost) position of the second piston; and
a second outlet gas port, which may be integral with or separate from the second inlet gas port, provided in the lower portion of the second cylinder (in the wall, alternatively, in the bottom end) of the second cylinder, below a bottommost (lowermost) position of the second piston.

11. The pneumatic cylinder medical ventilator of claim 9, wherein:
the first piston comprises a disc-like structure having an outer diameter (OD) corresponding with the diameter (ID) of the first cylinder; and
the second piston comprises a disc-like structure having an outer diameter (OD) corresponding with the diameter (ID) of the second cylinder.

12. The pneumatic cylinder medical ventilator of claim 9, further comprising:
means for creating an air-tight seal between the first piston and the first cylinder; and
means for creating an air-tight seal between the second piston and the second cylinder;

13. The pneumatic cylinder medical ventilator of claim 9, wherein:
the second diameter (bore of the second cylinder) is greater than the first diameter (bore of the first cylinder).

14. The pneumatic cylinder medical ventilator of claim 9, wherein:
the second piston has a longer stroke than the first piston.

15. The pneumatic cylinder medical ventilator of claim 9, further comprising:
a weight disposed on the link to impart a downward force to the first and second pistons.

16. The pneumatic cylinder medical ventilator of claim 9, further comprising:
first stops, defining a lower limit for movement of the pistons;
second stops limiting the upward motion of the pistons.

17. The pneumatic cylinder medical ventilator of claim 9, further comprising:
a first valve associated with the first cylinder; and
a second valve associated with the second cylinder.

18. A pneumatic cylinder medical ventilator system comprising:
the pneumatic cylinder medical ventilator of claim 9; and
a source of pressurized air comprising either (i) a first supply of air (or oxygen), such as may be supplied in a hospital room, such as at a pressure of 50-55 psi, or (ii) a second supply of air (or oxygen), such as may be supplied in a stand-alone tank, such as at a pressure of 200 psi (regulator included).

19. The pneumatic cylinder medical ventilator system of claim 18, further comprising:
a first regulator valve, receiving the pressurized air; and
a second regulator valve, controlling flow of air out of the first cylinder.

20. The pneumatic cylinder medical ventilator system of claim 18, further comprising:
a patient circuit selectively connected to (i) the inlet gas port of the second cylinder (FIG. 2A) and (ii) the outlet gas port of the first cylinder (FIG. 2B).
